(19) United States
(12) Patent Application Publication
(10) Pub. No.: US 2016/0131643 A1
(43) Pub. Date: May 12, 2016

(54) MICROFLUIDIC COLLABORATIVE ENZYME ENHANCED REACTIVE CEER IMMUNOASSAY

(71) Applicant: NESTEC S.A., Vevey (CH)

(72) Inventors: India Severin, Lausanne (CH); Katya Magonova, Tempe, AZ (US)

(73) Assignee: NESTEC S.A., Vevey (CH)

(21) Appl. No.: 14/988,643

(22) Filed: Jan. 5, 2016

Related U.S. Application Data
(63) Continuation of application No. PCT/EP2014/063846, filed on Jun. 30, 2014.
(60) Provisional application No. 61/744,308, filed on Sep. 24, 2012.

(51) Int. Cl. G01N 33/543; G01N 33/564; G01N 33/552

(52) U.S. Cl. CPC G01N 33/54393; G01N 33/54366; G01N 33/552; G01N 33/564; G01N 2333/904; G01N 2333/908

(57) ABSTRACT

The present invention provides methods for diagnosing a disease state in a patient by detecting the presence, expression level and/or activation level of a target analyte in a patient sample using a proximity dual detection assay on a microcarrier. The present invention also provides an assay device for performing the methods described herein.

### CROSS-REFERENCE TO RELATED APPLICATIONS

The present application is a continuation of PCT/EP2014/063846, filed Jun. 30, 2014, which application claims priority to U.S. Provisional Application No. 61/844,308, filed Jul. 9, 2013, the teachings of which are hereby incorporated by reference in their entireties for all purposes.

### BACKGROUND OF THE INVENTION

Traditional epidemiological studies of large cohorts of patients do not account for genetic variability of each individual within a given population. This results in a particular drug or therapeutic being very effective for some individuals, but not very effective for others. The goal of personalized medicine is to ensure a given drug is administered to the right individual at the right dose.

Various biomarkers such as genes, SNPs, enzyme activities, protein expression and activation as well as their impact on metabolic pathways can be used to develop optimization strategies for therapeutics. Identification of relevant biomarkers can be used to develop diagnostic assays that are useful in understanding drug efficacies and risks of side-effects. Choosing the proper dose or dosing schedule can be very important in optimizing a patient's response and minimizing a patent's risk.

Identification of the proper biomarker is an important first step in the development of diagnostic assays and kits for personalized medicine. Once the biomarker is identified, its presence or absence can be verified, and then quantified or measured using diagnostics tests. In certain instances, a point-of-care approach is used, where the assay is performed at a location on or near a site of patient care where medical testing and/or treatment can be performed. For example, locations for point-of-care may include, but are not limited to, hospitals, patient homes, a physician's office, or a site of an emergency.

The benefit of rapidly diagnosing a medical condition, selecting the proper therapy or optimizing the dose at the point-of-care has many advantages. By personalizing the drug, the dose and treatment regimen means the patient has the best chance of benefiting from modern medicine. What is needed in the art are new ways to identify and measure biomarkers for the clinical benefit of patients using a point-of-care device. The present invention satisfies this and other needs.

### BRIEF SUMMARY OF THE INVENTION

In one embodiment, the present invention provides a method such as an in vitro method, for performing a multiplexed immunoassay such as a microfluidic collaborative enzyme enhanced reactive (CEER) immunoassay, on a sample. The method comprises:
(a) contacting a cell lysate with a plurality of capture antibodies attached to an encoded microcarrier, specific for at least one analyte to form a plurality of captured analytes;
(b) contacting the plurality of captured analytes with at least two types of detection antibodies specific for the corresponding analytes, wherein at least one type of detection antibodies has a first member of a signal amplification pair, to form a plurality of detectable captured analytes;
(c) contacting the plurality of detectable captured analytes with a second member of the signal amplification pair to generate an amplified signal; and
(d) detecting the amplified signal generated from the first and second members of the signal amplification pair.

In some embodiments, the encoded microcarrier is made from a material selected from the group of latex, polystyrene, cross-linked dextrans, polymethylstyrene, polycarbonate, polypropylene, cellulose, polyacrylamide, polydimethylacrylamide, fluorinated ethylene-propylene, glass, SiO₂, silicon, PMMA (polymethylmethacrylate), gold, silver, aluminum, steel, and SU-8. In one embodiment, the encoded microcarrier is made of silicon.

In some embodiments, the encoded microcarrier is shaped as a member selected from the group of a sphere, a wafer, a square, a disk, a rectangle, a circle, a triangle and a hexagon. In one embodiment, the encoded microcarrier is shaped as a wafer.

In some embodiments, the encoded microcarrier comprises a plurality of microcarriers.

In some embodiments, the plurality of microcarriers has at least two populations. In some instances, each of the at least two populations of encoded microcarriers has different functions. In one instance, the specific function is identifiable by the code.

In some embodiments, the encoded microcarrier has a function, which is determined by reading the code. In some instances, the code is in the form of a configuration of traversing holes in the microcarrier. In another instance, the specific function is the identity of the plurality of capture antibodies.

In some embodiments, the sample is selected from the group of whole blood, serum, plasma, urine, sputum, bronchial lavage fluid, tears, nipple aspirate, lymph, saliva, cerebral spinal fluid (CSF), fine needle aspirate (FNA), and a combination thereof.

In another embodiment, the detection antibodies comprise:
(i) a plurality of activation state-independent antibodies labeled with a facilitating moiety, and
(ii) a plurality of activation state-dependent antibodies labeled with the first member of a signal amplification pair.

In some aspects, the facilitating moiety generates an oxidizing agent which channels to and reacts with the first member of the signal amplification pair. In certain instances, the facilitating moiety is glucose oxidase. In certain aspects, the oxidizing agent is hydrogen peroxide (H₂O₂). In some aspects, the first member of the signal amplification pair is a peroxidase. In certain instances, the peroxidase is horseradish peroxidase (HRP).

In some aspects, the second member of the signal amplification pair is a tyramide reagent. In certain instances, the tyramide reagent is biotin-tyramide.

In some embodiments, the amplified signal is generated by peroxidase oxidization of the biotin-tyramide to produce an activated tyramide. In some aspects, the activated tyramide is directly detected. In other aspects, the activated tyramide is detected upon the addition of a signal-detecting reagent. In some instances, the signal-detecting reagent is a streptavidin-labeled fluorophore. In other instances, the signal-detecting reagent is a combination of a streptavidin-labeled peroxidase and a chromogenic reagent. In some embodiments, the chromogenic reagent is 3,3′,5,5′-tetramethylbenzidine (TMB).

In some embodiments, the method provided herein is used as a human disease diagnostic test. In some aspects, the disease is a metabolic disease, a brain and cognitive disorder, a brain disorder, a cognitive disorder, a gastrointestinal disease or a cancer.

In some aspects, the metabolic disease is diabetes, including prediabetes, type 1 diabetes, type 2 diabetes, other forms of diabetes, and disorders associated with diabetes.

In some aspects, the brain and cognitive disorder is Alzheimer's disease.

In some instances, the cancer is a member selected from the group of breast cancer, colorectal cancer, gastrointestinal stromal tumors, gastrointestinal carcinoid tumors, colon cancer, rectal cancer, anal cancer, bile duct cancer, small intestine cancer, prostate cancer, and gastric cancer.

In some embodiments, the at least one analyte comprises at least one signal transduction molecule, a pathological protein, or an autoantibody.

In some instances, the at least one signal transduction molecule is selected from the group of AMP-activated protein kinase (AMPK), AMPK kinase (AMPKK), calmodulin-dependent protein kinase kinase (CAMKK), LKB1, transforming growth factor-β-activated kinase 1 (TAK1), AKT, adiponectin, leptin, glucose, other AMPK modulators, and a combination thereof.

In other instances, the at least one pathological protein is selected from the group of tau, activated (phosphorylated) tau, amyloid beta (Aβ) peptide, soluble amyloid precursor protein (APPβ), fragments thereof, complexes thereof, and oligomers thereof.

In other instances, the at least one autoantibody is selected from the group of GAD65 autoantibody, insulinoma-antigen 2 (IA-2) autoantibody, insulin autoantibody (IAA), zinc transporter protein 8 (ZnT8) autoantibody, and a combination thereof.

In yet other instances, the at least one signal transduction molecule is a receptor tyrosine kinase. In certain embodiments, the receptor tyrosine kinase is a member selected from the group of HER1, HER2, HER3, HER4, VEGFR-1, VEGFR-2, VEGFR-3, FLT-3, FLK-2, PDGFR, c-KIT, INSR, IGF-IR, IGF-IIR, IRR, CSF-1R, cMET and a combination thereof. In other embodiments, the at least one receptor tyrosine kinase further comprises a non-receptor tyrosine kinase.

In some embodiments, the sample flows through a microchannel comprising the encoded microcarrier. In some aspects, microchannel is transparent on at least one side.

In some embodiments, the amplified signal is correlated with the identity of the encoded microcarrier. In some instances, the amplified signal is correlated with a kinetic binding parameter. These parameters include, for example, detailed binding kinetic parameters such as an association rate, kₒₙ; a dissociation rate, k_{off}; or an affinity constant, Kₐ.

The methods herein are preferably in vitro methods. In one embodiment, the present invention provides an in vitro method for performing a multiplexed immunoassay such as a microfluidic collaborative enzyme enhanced reactive (CEER) immunoassay, on a sample, which comprises:
(a) contacting a cell lysate with a plurality of capture antibodies attached to an encoded microcarrier, specific for at least one analyte to form a plurality of captured analytes;
(b) contacting the plurality of captured analytes with at least two types of detection antibodies specific for the corresponding analytes, wherein at least one type of detection antibodies has a first member of a signal amplification pair, to form a plurality of detectable captured analytes;
(c) contacting the plurality of detectable captured analytes with a second member of the signal amplification pair to generate an amplified signal; and
(d) detecting the amplified signal generated from the first and second members of the signal amplification pair.

In some embodiments of the method, the detecting the amplified signal is performed by using an array of optical sensors coupled with optic means, wherein the sensor is a CCD or a C-MOS photo-sensor.

In another embodiment, the present invention provides a point of care diagnostic system. The system comprises:
(a) an assay device comprising a reaction chamber, wherein the reaction chamber comprises a microchannel; and
(b) a kit comprising:
   i) a first population of individually encoded microcarriers having a function, wherein the first population of individually encoded microcarriers has a first plurality of capture antibodies attached thereto; and
   ii) at least two types of detection antibodies comprising;
      a) a plurality of activation state-independent antibodies labeled with a facilitating moiety; and
      b) a plurality of activation state-dependent antibodies labeled with a first member of a signal amplification pair.

In some embodiments of the system, the device comprises a means to restrict the movement of the microcarriers in the longitudinal direction of the microchannel, while still letting fluids flow through. In other embodiments, the kit further comprises a second population of individually encoded microcarriers and the second population of individually encoded microcarriers has a second plurality of capture antibodies. In another embodiment, the first population of individually encoded microcarriers has the function and the second population of individually encoded microcarriers has a different function.

In some aspects, the function is the identity of the plurality of capture antibodies. In other aspects, the function is identifiable by the code. In some embodiments, the code is in the form of a configuration of traversing holes in the microcarrier.

In some embodiments, the encoded microcarriers are made from a material selected from the group of latex, polystyrene, cross-linked dextrans, polymethylstyrene, polycarbonate, polypropylene, cellulose, polyacrylamide, polydimethylacrylamide, fluorinated ethylene-propylene, glass, SiO₂, silicon, PMMA (polymethylmethacrylate), gold, silver, aluminum, steel, and SU-8. In one embodiment, the encoded microcarriers are made of silicon.

In some embodiments, the function of the encoded microcarrier is determined by reading the code.

In some embodiments, the encoded microcarrier is shaped as a member selected from the group of a sphere, a wafer, a square, a disk, a rectangle, a circle, a triangle and a hexagon. In one embodiment, the encoded microcarrier is shaped as a wafer.

These and other objects, features, and advantages of the present invention will become more apparent to one of skill in the art in view of the the following detailed description and figures.

### BRIEF DESCRIPTION OF THE DRAWINGS

FIGS. 1A-1B show the results from an exemplary embodiment of the invention. FIG. 1A shows the fluorescence from a dilution series of BT474 cell lysates when a high concentration (1:500) of biotin-tyramide (BT-Tyr) was used. FIG. 1B shows the fluorescence from a dilution series of BT474 cell lysates when a low concentration (1:1000) of BT-Tyr was used.

FIGS. 2A-2B show microscope images of the results depicted in FIG. 1B. FIG. 2A shows an image of microcarriers incubated with a low concentration (1:1000) of biotin-tyramide and a sample of 75 cells. FIG. 2B shows an image of microcarriers incubated with a low concentration (1:1000) of biotin-tyramide and a sample of 7.5 cells.

### DETAILED DESCRIPTION OF THE INVENTION

### I. INTRODUCTION

The present invention provides methods, devices, uses and kits for the rapid identification and quantification of an analyte such as a protein biomarker. In the multiplexed immunoassay of the present invention, a plurality of capture antibodies are attached to an encoded microcarrier to retain a biomarker from, for example, a cell lysate. The encoded microcarrier can be used to identify the particular capture antibody and thus identify the biomarker i.e., analyte captured. In certain instances, the invention provides an in vitro use of a multiplexed immunoassay to identify an analyte (e.g., a biomarker) for a diagnostic purpose such as identifying a patient with a disease, or whether a patient will respond to therapy, or the proper dose of a drug to administer to a patient. These purposes can be achieved by measuring the expression and or activation levels of the analyte identified.

Using the present invention, it is possible to rapidly and accurately diagnose medical conditions such as metabolic disease, brain and cognitive disease, gastrointestinal disease, or cancer, identify the proper therapy and optimize the therapeutic dose, which provides significant benefits to patients, and care-practitioners.

Advantageously, the methods, devices, uses and kits of the present invention facilitate diagnostic testing that can be delivered at the point-of-care, which is the site where real time or near real time diagnostic testing can be done so that the resulting test is performed more efficiently than comparable tests that do not employ the present system. Point-of-care diagnostic testing is testing at or near the site of patient care, such as a physician's office or hospital or wherever medical care is provided. A rapid turnaround time for assay results provides many benefits including real time evidence-based decisions, immediate treatment of patients, minimization of unnecessary tests, minimization of side-effects and significant cost efficiencies.

In certain instances, the methods and devices of the present invention enable laboratory tests to be performed in immediate proximity to the patient, outside of a central laboratory. In certain aspects, the methods use a sample, which is whole blood, plasma, serum, a fine needle aspirate (FNA), or cerebral spinal fluid (CSF), and ready-to-use reagents, as well as disposable assay cartridges. In certain aspects, the inventive devices are portable and use individual sample quantities and amounts.

### II. DEFINITIONS

As used herein, the following terms have the meanings ascribed to them unless specified otherwise.

The term “analyte” includes any molecule of interest, typically a macromolecule such as a polypeptide, whose presence, amount, and/or identity is determined. In certain instances, the analyte is a cellular component of circulating cells of a solid tumor or in cerebral spinal fluid, peripheral blood or serum. Preferably, the analyte or biomarker is a signal transduction molecule, a pathological molecule, or an autoantibody.

The term “signal transduction molecule” or “signal transducer” includes proteins and other molecules that carry out the process by which a cell converts an extracellular or intracellular signal or stimulus into a response, typically involving ordered sequences of biochemical reactions inside the cell. Examples of signal transduction molecules include, but are not limited to, receptor tyrosine kinases such as EGFR (e.g., EGFR/HER-1/ErbB1, HER-2/Neu/ErbB2, HER-3/ErbB3, HER-4/ErbB4), VEGFR-1/FLT-1, VEGFR-2/FLK-1/KDR, VEGFR-3/FLT-4, FLT-3/FLK-2, PDGFR (e.g., PDGFRA, PDGFRB), c-KIT/SCFR, INSR (insulin receptor), IGF-IR, IGF-IIR, IRR (insulin receptor-related receptor), CSF-1R, FGFR 1-4, HGFR 1-2, CCK4, TRK A-C, MET, RON, EPHA 1-8, EPHB 1-6, AXL, MER, TYRO3, TIE 1-2, TEK, RYK, DDR 1-2, RET, c-ROS, V-cadherin, LTK (leukocyte tyrosine kinase), ALK (anaplastic lymphoma kinase), ROR 1-2, MUSK, AATYK 1-3, RTK 106, and truncated forms of the receptor tyrosine kinases such as p95ErbB2; non-receptor tyrosine kinases such as BCR-ABL, Src, Frk, Btk, Csk, Abl, Zap70, Fes/Fps, Fak, Jak, Ack, and LIMK; tyrosine kinase signaling cascade components such as AKT, MAPK/ERK, MEK, RAF, PLA2, MEKK, JNKK, JNK, p38, Shc (p66), PI3K, Ras (e.g., K-Ras, N-Ras, H-Ras), Rho, Rac1, Cdc42, PLC, PKC, p70 S6 kinase, p53, cyclin D1, STAT1, STAT3, PIP2, PIP3, PDK, mTOR, BAD, p21, p27, ROCK, IP3, TSP-1, NOS, PTEN, RSK 1-3, JNK, c-Jun, Rb, CREB, Ki67, and paxillin; nuclear hormone receptors such as estrogen receptor (ER), progesterone receptor (PR), androgen receptor, glucocorticoid receptor, mineralocorticoid receptor, vitamin A receptor, vitamin D receptor, retinoid receptor, thyroid hormone receptor, and orphan receptors; nuclear receptor coactivators and repressors such as amplified in breast cancer-1 (AIB1) and nuclear receptor corepressor 1 (NCOR), respectively; and a combination thereof. Additional examples of signal transduction molecules, include but are not limited to, AMP-activated protein kinase (AMPK), AMPK kinase (AMPKK), calmodulin-dependent protein kinase kinase (CAMKK), LKB1, transforming growth factor-β-activated kinase 1 (TAK1), AKT, adiponectin, leptin, glucose, glycogen, AMP, and ATP.

The term “pathological molecule” includes proteins and other molecules associated with the pathology of a disease or disorder such as a neurodegenerative disease including Alzheimer's disease, Parkinson's disease, Huntington's disease, dementia, amyotrophic lateral sclerosis (ALS), spinocerebellar ataxia, spinal muscular atrophy, and the like.

The term “capture antibody” is intended to include an immobilized antibody which is specific for (i.e., binds, is bound by, or forms a complex with) one or more analytes of interest in a sample such as a cellular extract. In preferred embodiments, the capture antibody is restrained on a microcarrier (e.g., solid support) in an array device. Suitable capture antibodies for immobilizing any of a variety of signal transduction molecules on a solid support are available from Upstate (Temecula, Calif.), Biosource (Camarillo, Calif), Cell Signaling Technologies (Danvers, Mass.), R&D Systems (Minneapolis, Minn.), Lab Vision (Fremont, Calif.), Santa Cruz Biotechnology (Santa Cruz, Calif.), Sigma (St. Louis, Mo.), and BD Biosciences (San Jose, Calif.).

The term “detection antibody” as used herein includes an antibody comprising a detectable label which is specific for (i.e., binds, is bound by, or forms a complex with) one or more analytes of interest in a sample. The term also encompasses an antibody which is specific for one or more analytes of interest, wherein the antibody can be bound by another species that comprises a detectable label. Examples of detectable labels include, but are not limited to, biotin/streptavidin labels, nucleic acid (e.g., oligonucleotide) labels, chemically reactive labels, fluorescent labels, enzyme labels, radioactive labels, and a combination thereof Suitable detection antibodies for detecting the activation state and/or total amount of any of a variety of signal transduction molecules are available from Upstate (Temecula, Biosource (Camarillo, Calif.), Cell Signaling Technologies (Danvers, Mass.), R&D Systems (Minneapolis, Minn.), Lab Vision (Fremont, Calif.), Santa Cruz Biotechnology (Santa Cruz, Calif), Sigma (St. Louis, Mo.), and BD Biosciences (San Jose, Calif).

The term “activation state-dependent antibody” includes a detection antibody which is specific for (i.e., binds, is bound by, or forms a complex with) a particular activation state of one or more analytes of interest in a sample. In preferred embodiments, the activation state-dependent antibody detects the phosphorylation, ubiquitination, and/or complexation state of one or more analytes such as one or more signal transduction molecules. In some embodiments, the phosphorylation of members of the EGFR family of receptor tyrosine kinases and/or the formation of heterodimeric complexes between EGFR family members is detected using activation state-dependent antibodies. Non-limiting examples of activation states (listed in parentheses) that are suitable for detection with activation state-dependent antibodies include: EGFR (EGFRvIII, phosphorylated (p−) EGFR, EGFR:Shc, ubiquitinated (u−) EGFR, p-EGFRvIII); ErbB2 (p95:truncated (Tr)-ErbB2, p-ErbB2, p95:Tr-p-ErbB2, HER-2:Shc, ErbB2:PI3K, ErbB2:EGFR, ErbB2:ErbB3, ErbB2:ErbB4); ErbB3 (p-ErbB3, ErbB3:PI3K, p-ErbB3:PI3K, ErbB3:Shc); ErbB4 (p-ErbB4, ErbB4:Shc); ER (p-ER (S118, S167); IGF-IR (p-IGF-1R, IGF-1R:IRS, IRS:PI3K, p-IRS, IGF-1R:PI3K); INSR (p-INSR); KIT (p-KIT); FLT3 (p-FLT3); HGFRI (p-HGFRI); HGFR2 (p-HGFR2); RET (p-RET); PDGFRa (p-PDGFRa); PDGFRP (p-PDGFRP); VEGFRI (p-VEGFRI, VEGFRI:PLCg, VEGFR1:Src); VEGFR2 (p-VEGFR2, VEGFR2:PLCy, VEGFR2:Src, VEGFR2:heparin sulphate, VEGFR2:VE-cadherin); VEGFR3 (p-VEGFR3); FGFR1 (p-FGFR1); FGFR2 (p-FGFR2); FGFR3 (p-FGFR3); FGFR4 (p-FGFR4); Tie1 (p-Tie1); Tie2 (p-Tie2); EphA (p-EphA); EphB (p-EphB); NFKB and/or IKB (p-IK (S32), p-NFKB (S536), p-P65:IKBa); Akt (p-Akt (T308, S473)); PTEN (p-PTEN); Bad (p-Bad (S112, S 136), Bad:14-3-3); mTor (p-mTor (S2448)); p70S6K (p-p70S6K (T229, T389)); Mek (p-Mek (S217, S221)); Erk (p-Erk (T202, Y204)); Rsk-1 (p-Rsk-1 (T357, S363)); Jnk (p-Jnk (T183, Y185)); P38 (p-P38 (T180, Y182)); Stat3 (p-Stat-3 (Y705, S727)); Fak (p-Fak (Y576)); Rb (p-Rb (S249, T252, S780)); Ki67; p53 (p-p53 (5392, S20)); CREB (p-CREB (S133)); c-Jun (p-c-Jun (S63)); cSrc (p-cSrc (Y416)); paxillin (p-paxillin (Y118)); AMPK (p-AMPK (T172, T183); and tau (p-tau (Y18, Y29, S46, T181, S199, S202, T205, T212, S214, T217, T231, S235, S262, S356, S396, S400, S404, S412, S422, S470, S492, S498, S515, S516, S519, T534, T548, S552, S622, S641, S713, S721, S726, T739, etc.)

The term “activation state-independent antibody” includes a detection antibody which is specific for (i.e., binds, is bound by, or forms a complex with) one or more analytes of interest in a sample irrespective of their activation state. For example, the activation state-independent antibody can detect both phosphorylated and unphosphorylated forms of one or more analytes such as one or more signal transduction molecules.

The term “nucleic acid” or “polynucleotide” includes deoxyribonucleotides or ribonucleotides and polymers thereof in either single- or double-stranded form such as, for example, DNA and RNA. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs include, without limitation, phosphorothioates, phosphoramidates, methyl phosphonates, chiral-methyl phosphonates, 2′-O-methyl ribonucleotides, and peptide-nucleic acids (PNAs). Unless specifically limited, the term encompasses nucleic acids containing known analogues of natural nucleotides that have similar binding properties as the reference nucleic acid. Unless otherwise indicated, a particular nucleic acid sequence also implicitly encompasses conservatively modified variants thereof and complementary sequences as well as the sequence explicitly indicated.

The term “oligonucleotide” refers to a single-stranded oligomer or polymer of RNA, DNA, RNA/DNA hybrid, and/or a mimetic thereof. In certain instances, oligonucleotides are composed of naturally-occurring (i.e., unmodified) nucleobases, sugars, and internucleoside (backbone) linkages. In certain other instances, oligonucleotides comprise modified nucleobases, sugars, and/or internucleoside linkages.

The term “incubating” is used synonymously with “contacting” and “exposing” and does not imply any specific time or temperature requirements unless otherwise indicated.

As used herein, the term “dilution series” is intended to include a series of descending concentrations of a particular sample (e.g., cell lysate) or reagent (e.g., antibody). A dilution series is typically produced by a process of mixing a measured amount of a starting concentration of a sample or reagent with a diluent (e.g., dilution buffer) to create a lower concentration of the sample or reagent, and repeating the process enough times to obtain the desired number of serial dilutions. The sample or reagent can be serially diluted at least 2, 3, 4, 5, 6, 7, 8, 9, 10, 15, 20, 25, 30, 35, 40, 45, 50, 100, 500, or 1000-fold to produce a dilution series comprising at least 2, 3, 4, 5, 6, 7, 8, 9, 10, 11, 12, 13, 14, 15, 16, 17, 18, 19, 20, 25, 30, 35, 40, 45, or 50 descending concentrations of the sample or reagent. For example, a dilution series comprising a 2-fold serial dilution of a capture antibody reagent at a 1 mg/ml starting concentration can be produced by mixing an amount of the starting concentration of capture antibody with an equal amount of a dilution buffer to create a 0.5 mg/ml concentration of the capture antibody, and repeating the process to obtain capture antibody concentrations of 0.25 mg/ml, 0.125 mg/ml, 0.0625 mg/ml, 0.0325 mg/ml, etc.

The term “superior dynamic range” as used herein refers to the ability of an assay to detect a specific analyte in as few as one cell or in as many as thousands of cells. For example, the immunoassays described herein possess superior dynamic range because they advantageously detect a particular signal transduction molecule of interest in about 1-10,000 cells (e.g., about 1, 5, 10, 25, 50, 75, 100, 250, 500, 750, 1000, 2500, 5000, 7500, or 10,000 cells) using a dilution series of capture antibody concentrations.

The term “microcarrier” as used herein is a microparticle or micro-scale solid support that is functionalized on at least one surface, thereby allowing it to be used to analyze and/or to react with an analyte in a sample. The term “functionalized microcarrier” is used simultaneously herein. A “set of microcarriers” refers to one or more microcarriers used to capture and/or detect one specific target analyte. A set may be only one microcarrier or more than one microcarrier. The microcarriers of one set may carry more than one capture molecule (e.g., capture antibody or capture ligand) in order to capture two or more molecules of the target analyte, but this is still referred to as one function. Two different sets of microcarriers, that are distinguishable from each other, have different capture molecules bound to their surfaces.

The term “code” as used herein is any attribute or characteristic of a microcarrier that is distinguishable upon observation or detection and that is used to identify the microcarrier or to associate the microcarrier to a specific population (e.g., the population of microcarriers having a given function). A code on a microcarrier can be determined independently of its position and independently of the performance of an assay, i.e. it does not require the presence of a target analyte to be revealed. Typically, a code is characterized by the optical or magnetic response of the microcarrier upon observation. This response might be defined for the microcarrier as a whole (e.g. the color of the microcarrier) or might be spatially modulated in or on the microcarrier to result in a patterned layout (e.g. a barcode obtained by the modulation of the color on the microcarrier). Examples of codes include, but are not limited to, color, shape, size, imprinted or engraved patterns, configuration of holes, holographic patterns, magnetic signatures, chemical composition, modification of light transmission or reflection characteristics, quantum dots emission, distinctive detectable foreign objects (e.g. oligonucleotide or other polymers), an IC chip attached or embedded to/in the surface.

The term “encoded microcarrier” refers to a microcarrier that has a code. The microcarriers are individually encoded, i.e., each microcarrier carries its own code, even if several microcarriers (typically the microcarriers of one set) may carry a code with a same value (i.e., the microcarriers are not distinguishable based on their code alone). The different sets of encoded microcarriers can be distinguished and/or identified independently of the position of the microcarriers in the microchannel and independently of the performance of an assay.

The term “reaction chamber” refers to the space where the capturing and/or detecting reaction occurs between the target analyte and the capture molecule (e.g., capture antibody or capture ligand) and/or detection antibody.

As used herein, the term “circulating cells” comprises extratumoral cells that have either metastasized or micrometastasized from a solid tumor. Examples of circulating cells include, but are not limited to, circulating tumor cells, cancer stem cells, and/or cells that are migrating to the tumor (e.g., circulating endothelial progenitor cells, circulating endothelial cells, circulating pro-angiogenic myeloid cells, circulating dendritic cells, etc.).

The term “sample” as used herein includes any biological specimen obtained from a patient. Samples include, without limitation, whole blood, plasma, serum, red blood cells, white blood cells (e.g., peripheral blood mononuclear cells), cerebral spinal fluid, ductal lavage fluid, nipple aspirate, lymph (e.g., disseminated tumor cells of the lymph node), bone marrow aspirate, saliva, urine, stool (i.e., feces), sputum, bronchial lavage fluid, tears, fine needle aspirate (e.g., harvested by random periareolar fine needle aspiration), any other bodily fluid, a tissue sample (e.g., tumor tissue) such as a biopsy of a tumor (e.g., needle biopsy) or a lymph node (e.g., sentinel lymph node biopsy), and cellular extracts thereof. In some embodiments, the sample is whole blood or a fractional component thereof such as plasma, serum, or a cell pellet. In other embodiments, the sample is obtained by isolating circulating cells of a solid tumor from whole blood or a cellular fraction thereof using any technique known in the art. In other embodiments, the sample is a formalin fixed paraffin embedded (FFPE) tissue sample, e.g., from a solid tumor of the breast.

A “biopsy” refers to the process of removing a tissue sample for diagnostic or prognostic evaluation, and to the tissue specimen itself. Any biopsy technique known in the art can be applied to the methods and compositions of the present invention. The biopsy technique applied will generally depend on the tissue type to be evaluated and the size and type of the tumor (i.e., solid or suspended (i.e., blood or ascites)), among other factors. Representative biopsy techniques include excisional biopsy, incisional biopsy, needle biopsy (e.g., core needle biopsy, fine-needle aspiration biopsy, etc.), surgical biopsy, and bone marrow biopsy. Biopsy techniques are discussed, for example, in Harrison's Principles of Internal Medicine, Kasper, et al., eds., 16th ed., 2005, Chapter 70, and throughout Part V. One skilled in the art will appreciate that biopsy techniques can be performed to identify cells of interest in a given tissue sample.

The term “subject” or “patient” or “individual” typically includes humans, but can also include other animals such as, e.g., other primates, rodents, canines, felines, equines, ovines, porcines, and the like.

The present invention is useful in a point-of-care location or a laboratory-based clinical chemistry testing location. As used herein, a point-of-care (POC) location means hospital testing, bedside testing, emergency room, physician's office, clinic, blood bank, operating room, home, laboratory or nursing home testing, remote testing, and the like. It is particularly useful when embodied in an analytical system or device where collection of the sample and performance of the test or assay, occur at the same location, as in point-of-care testing. However, it is understood that in addition to a clinical testing laboratory, the invention may be used in any laboratory.

The terms “metabolic disease,” “metabolic disorder” and “metabolic syndrome” are herein used interchangeably and refer to any of the conditions caused by a disruption in normal metabolism (e.g., metabolic balance). Metabolism is the process of converting food to energy on a cellular level. Typically, metabolic disease is characterized by increased blood pressure, a high blood sugar level, excess body fat in the abdominal area, insulin resistance, glucose intolerance, and/or an abnormal cholesterol level. Non-limiting examples of metabolic disease/disorder include type 1 diabetes, type 2 diabetes, pre-diabetes, diabetes associated diseases, hyperinsulinemia, lysosomal storage disorders, Gaucher's disease, impaired glucose tolerance, impaired fasting glycemia, insulin resistance, and dyslipidemia.

The term “brain and cognitive disease” or “brain and cognitive disorder” refers to a neurological and/or mental health disease/disorder that primarily affects learning, memory, perception and problem solving. The term includes delirium, major and mild neurocognitive disorder, dementia, Alzheimer's disease, Parkinson's disease, Huntington's disease, amytrophic lateral sclerosis (ALS), spinocerebellar ataxia, spinal muscular atrophy, and all known neurological diseases/disorders characterized by a decline in a person's mental ability that negatively interferes with daily life.

The term “cancer” is intended to include any member of a class of diseases characterized by the uncontrolled growth of aberrant cells. The term includes all known cancers and neoplastic conditions, whether characterized as malignant, benign, soft tissue, or solid, and cancers of all stages and grades including pre- and post-metastatic cancers. Examples of different types of cancer include, but are not limited to, breast cancer; lung cancer (e.g., non-small cell lung cancer); digestive and gastrointestinal cancers such as colorectal cancer, gastrointestinal stromal tumors, gastrointestinal carcinoid tumors, colon cancer, rectal cancer, anal cancer, bile duct cancer, small intestine cancer, and stomach (gastric) cancer; esophageal cancer; gallbladder cancer; liver cancer; pancreatic cancer; appendix cancer; ovarian cancer; renal cancer (e.g., renal cell carcinoma); cancer of the central nervous system; skin cancer; lymphomas; choriocarcinomas; head and neck cancers; osteogenic sarcomas; and blood cancers. As used herein, a “tumor” comprises one or more cancerous cells.

### III. DETAILED DESCRIPTIONS OF EMBODIMENTS

In one aspect, the invention provides a method for diagnosing cancer, metabolic disease, gastrointestinal disease or brain and cognitive disease, brain disease, or cognitive disease by performing a multiplexed assay based on a proximity dual detection assay on microcarriers in an assay device. The method comprises: a) contacting a patient sample with a plurality of encoded microcarriers positioned or immobilized in a microchannel of an assay device to form a plurality of captured analytes; b) contacting the plurality of captured analytes on the microcarriers with at least two types of detection antibodies, wherein at least one type of detection antibodies has a member of a signal amplification pair, to form a plurality to detectable captured analytes; c) contacting the plurality of detectable captured analytes with a second member of the signal amplification pair to generate an amplified signal; and d) detecting the amplified signal generated from the first and second members of the signal amplification pair.

The presence, expression (e.g., total amount) levels and/or activation (e.g., phosphorylation) levels of the target analytes in a patient sample are used to determine whether the patient has a metabolic disease, a gastrointestinal disease, a brain and cognitive disease, or a cancer.

In some instances, the presence, expression (e.g., total amount) level and/or activation (e.g., phosphorylation) level of one or more signal transduction molecule such as HER1, HER2, HER3. HER4, VEGFR-1, VEGFR-2, VEGFR-3, FLT-3, FLK-2, PDGR, c-KIT, INSR, IGF-IR, IGF-IIR, IRR, CSF-IR, cMET, and a combination thereof is used to diagnose cancer in a patient.

In other instances, the presence, expression (e.g., total amount) level and/or activation (e.g., phosphorylation) levesl of one or more signal transduction molecule such as AMP-activated protein kinase (AMPK), AMPK kinase (AMPKK), calmodulin-dependent protein kinase kinase (CAMKK), LKB1, transforming growth factor-β-activated kinase 1 (TAK1), AKT, adiponectin, leptin, glucose, other AMP modulators, and a combination thereof is used to diagnose diabetes in a patient.

In other instances, the presence and/or expression (e.g., total amount) level of one or more autoantibody such as GAD65 autoantibody, insulinoma-antigen 2 (IA-2) autoantibody, insulin autoantibody (IAA), zinc transporter protein 8 (ZnT8) autoantibody and a combination thereof is used to diagnose diabetes in a patient.

In other instances, the presence, expression (e.g., total amount) level and/or activation (e.g., phosphorylation) level of one or more pathological molecule such as tau, amyloid beta (Aβ) protein, amyloid precursor protein (APP), amyloid beta (Aβ) peptides (e.g., β-amyloid protein 28, β-amyloid protein 40, β-amyloid protein 42, β-amyloid protein 43, soluble amyloid precursor protein beta (sAPPβ), and the like), any Aβ protein fragments, VILIP-1, and a combination thereof is used to diagnose Alzheimer's disease in a patient.

In still other instances, the presence or level of at least one cytokine or a plurality of cytokines in a sample is particularly useful in the present invention. As used herein, the term “cytokine” includes any of a variety of polypeptides or proteins secreted by immune cells that regulate a range of immune system functions and encompasses small cytokines such as chemokines. The term “cytokine” also includes adipocytokines, which comprise a group of cytokines secreted by adipocytes that function, for example, in the regulation of body weight, hematopoiesis, angiogenesis, wound healing, insulin resistance, the immune response, and the inflammatory response. Cytokines can be used to as a biomarker of disease, dysregulation or dysfunction.

In certain aspects, the presence or level of at least one cytokine includes, but is not limited to, TNF-α, TNF-related weak inducer of apoptosis (TWEAK), osteoprotegerin (OPG), IFN-α, IFN-β, IFN-γ, IL-1α, IL-1β, IL-1 receptor antagonist (IL-1ra), IL-2, IL-4, IL-5, IL-6, soluble IL-6 receptor (sIL-6R), IL-7, IL-8, IL-9, IL-10, IL-12, IL-13, IL-15, IL-17, IL-23, and IL-27 is determined in a sample. In certain other aspects, the presence or level of at least one chemokine such as, for example, CXCL1/GRO1/GROα, CXCL2/GRO2, CXCL3/GRO3, CXCL4/PF-4, CXCL5/ENA-78, CXCL6/GCP-2, CXCL7/NAP-2, CXCL9/MIG, CXCL10/IP-10, CXCL11/1-TAC, CXCL12/SDF-1, CXCL13/BCA-1, CXCL14/BRAK, CXCL15, CXCL16, CXCL17/DMC, CCL1, CCL2/MCP-1, CCL3/MIP-1α, CCL4/MIP-1β, CCL5/RANTES, CCL6/C10, CCL7/MCP-3, CCL8/MCP-2, CCL9/CCL10, CCL11/Eotaxin, CCL12/MCP-5, CCL13/MCP-4, CCL14/HCC-1, CCL15/MIP-5, CCL16/LEC, CCL17/TARC, CCL18/MIP-4, CCL19/MIP-3β, CCL20/MIP-3α, CCL21/SLC, CCL22/MDC, CCL23/MPIF1, CCL24/Eotaxin-2, CCL25/TECK, CCL26/Eotaxin-3, CCL27/CTACK, CCL28/MEC, CL1, CL2, and CX₃CL1 is determined in a sample.

In certain further aspects, the presence or level of at least one adipocytokine including, but not limited to, leptin, adiponectin, resistin, active or total plasminogen activator inhibitor-1 (PAI-1), visfatin, and retinol binding protein 4 (RBP4) is determined in a sample.

In other instances, the methods and systems of the present invention can be used to determine the presence or level of one or more growth factors in a sample. As used herein, the term “growth factor” includes any of a variety of peptides, polypeptides, or proteins that are capable of stimulating cellular proliferation and/or cellular differentiation.

In certain aspects, the presence or level of at least one growth factor including, but not limited to, epidermal growth factor (EGF), heparin-binding epidermal growth factor (HB-EGF), vascular endothelial growth factor (VEGF), pigment epithelium-derived factor (PEDF; also known as SERPINF1), amphiregulin (AREG; also known as schwannoma-derived growth factor (SDGF)), basic fibroblast growth factor (bFGF), hepatocyte growth factor (HGF), transforming growth factor-α (TGF-α), transforming growth factor-β (TGF-β), bone morphogenetic proteins (e.g., BMP1-BMP15), platelet-derived growth factor (PDGF), nerve growth factor (NGF), β-nerve growth factor (β-NGF), neurotrophic factors (e.g., brain-derived neurotrophic factor (BDNF), neurotrophin 3 (NT3), neurotrophin 4 (NT4), etc.), growth differentiation factor-9 (GDF-9), granulocyte-colony stimulating factor (G-CSF), granulocyte-macrophage colony stimulating factor (GM-CSF), myostatin (GDF-8), erythropoietin (EPO), and thrombopoietin (TPO) is determined in a sample. In one aspect, the presence or level of EGF, VEGF, PEDF, amphiregulin (SDGF), and/or BDNF is determined.

Various kinetic binding parameters can be determined. These include, for example, binding kinetic parameters (e.g., association rate, kₒₙ; dissociation rate, k_{off}; and affinity constant, Kₐ). These parameters can be used to confirm and measure direct molecular interactions, such as for example, receptor-ligand interactions, enzyme-substrate interactions, protein-nucleic acid interactions, protein-antibody interactions, and protein-protein interactions.

The step of detecting the amplified signal includes performing a fluorescence readout and correlating the fluorescence intensity with the level (e.g., amount, presence, absence, concentration) of the target analyte in the patient sample. The step can also include identifying the microcarrier. In some embodiments, fluorescence detection is performed using an optical sensor such as, but not limited to, a CCD or a C-MOS photo-sensor.

One of skill in the art will recognize that in a multiplexed assay steps (a) to (c) are performed in such a way to detect more than one specific target analyte. For example, the multiplex assay of the invention measures the levels of at least two different target analytes.

In some embodiments, the methods of the invention are performed in a microfluidic instrument, such as the Dynamic Multi-Analyte Technology (DMAT) machine (Biocartis, Mechelen, Belgium).

A. Analyte Detection Assays

Detailed descriptions of a single detection assay and a proximity dual detection assay are found in, for example, U.S. Pat. No. 8,163,499, which is herein incorporated by reference in its entirety for all purposes.

1. Single Detection Assays

In some embodiments, the assay for detecting the activation state of a particular analyte (e.g., signal transduction molecule or a neurodegenerative molecule) of interest in a cellular extract, which includes an analyte of interest implicated in a metabolic disease, or a brain of cognitive disorder, a neurological and/or mental health disorder or cancer. In certain instances, the analyte can be within tumor cells such as circulating cells of a solid tumor. Quite advantageously, the present invention provides a multiplex, high-throughput two-antibody assay having superior dynamic range. As a non-limiting example, the two antibodies used in the assay can comprise: (1) a capture antibody specific for the analyte; and (2) a detection antibody specific for an activated form of the analyte (i.e., activation state-dependent antibody). The activation state-dependent antibody is capable of detecting, for example, the phosphorylation, ubiquitination, and/or complexation state of the analyte. Alternatively, the detection antibody comprises an activation state-independent antibody, which detects the total amount of the analyte in the cellular extract. The activation state-independent antibody is generally capable of detecting both the activated and non-activated forms of the analyte.

In a preferred embodiment, the two-antibody assay comprises: (i) incubating a cellular extract with a plurality of dilution series of capture antibodies to form a plurality of captured analytes; (ii) incubating the plurality of captured analytes with activation state-dependent antibodies specific for the corresponding analytes to form a plurality of detectable captured analytes; (iii) incubating the plurality of detectable captured analytes with first and second members of a signal amplification pair to generate an amplified signal; and (iv) detecting the amplified signal generated from the first and second members of the signal amplification pair.

The two-antibody assays described herein are typically antibody-based arrays, which comprise a plurality of different capture antibodies at a range of capture antibody concentrations that are coupled to the surface of a microcarrier.

The capture antibodies and detection antibodies are preferably selected to minimize competition between them with respect to analyte binding (i.e., both capture and detection antibodies can simultaneously bind their corresponding signal transduction molecules or neurodegenerative molecules).

As such, in one embodiment, the present invention provides a method for performing a multiplexed immunoassay on a sample, the method comprising:
(a) contacting a cell lysate with a plurality of capture antibodies attached to an encoded microcarrier, specific for at least one analyte to form a plurality of captured analytes;
(b) contacting the plurality of captured analytes with at least one type of detection antibody specific for the corresponding analytes, wherein the at least one type of detection antibodies has a first member of a signal amplification pair, to form a plurality of detectable captured analytes;
(c) contacting the plurality of detectable captured analytes with a second member of the signal amplification pair to generate an amplified signal; and
(d) detecting the amplified signal generated from the first and second members of the signal amplification pair.

In certain aspects, the detection antibody comprises one type of detection antibody, which is a plurality of detection antibodies labeled with a first member of a signal amplification pair, specific for the at least one analyte. A facilitating moiety generates an oxidizing agent which reacts with the first member of the signal amplification pair. In certain aspects, the facilitating moiety is glucose oxidase (GO). GO can be attached to a substrate such as dextran and not an antibody. Multiple GO moieties can be attached to dextran.

In one embodiment, the detection antibodies comprise a first member of a binding pair (e.g., biotin) and the first member of the signal amplification pair comprises a second member of the binding pair (e.g., streptavidin). The binding pair members can be coupled directly or indirectly to the detection antibodies or to the first member of the signal amplification pair using methods well-known in the art. In certain instances, the first member of the signal amplification pair is a peroxidase (e.g., horseradish peroxidase (HRP), catalase, chloroperoxidase, cytochrome c peroxidase, eosinophil peroxidase, glutathione peroxidase, lactoperoxidase, myeloperoxidase, thyroid peroxidase, deiodinase, etc.), and the second member of the signal amplification pair is a tyramide reagent (e.g., biotin-tyramide).

Alternatively, the first member of the signal amplification pair can be directly bound to the detection antibody. In certain instances, the amplified signal is generated by peroxidase oxidation of the tyramide reagent to produce an activated tyramide in the presence of hydrogen peroxide (H₂O₂).

In one aspect, hydrogen peroxide (H₂O₂) is generated in situ. For example, a facilitating moiety such as glucose oxidase (GO), can be introduced into the immunoassay unattached to an antibody. That is, the GO can be attached to a substrate such as dextran. Multiple GO moieties can be attached to dextran. In the presence of glucose, the glucose oxidase generates an oxidizing agent such as hydrogen peroxide, which reacts with the first member of the signal amplification pair (e.g., HRP).

The activated tyramide is either directly detected or detected upon the addition of a signal-detecting reagent such as, for example, a streptavidin-labeled fluorophore or a combination of a streptavidin-labeled peroxidase and a chromogenic reagent. Examples of fluorophores suitable for use in the present invention include, but are not limited to, an Alexa Fluor®dye (e.g., Alexa Fluor®555, Alexa Fluor®647), fluorescein, fluorescein isothiocyanate (FITC), Oregon Green™rhodamine, Texas red, tetrarhodamine isothiocynate (TRITC), a CyDye™fluor (e.g., Cy2, Cy3, Cy5), and the like. The streptavidin label can be coupled directly or indirectly to the fluorophore or peroxidase using methods well-known in the art. Non-limiting examples of chromogenic reagents suitable for use in the present invention include 3,3′,5,5′-tetramethylbenzidine (TMB), 3,3′-diaminobenzidine (DAB), 2,2′-azino-bis(3-ethylbenzothiazoline-6-sulfonic acid) (ABTS), 4-chloro-1-napthol (4CN), and/or porphyrinogen.

In another embodiment, the present invention provides kits for performing the two-antibody assays described above comprising: (a) a dilution series of a plurality of capture antibodies restrained on a microcarrier; and (b) a plurality of detection antibodies (e.g., activation state-independent antibodies and/or activation state-dependent antibodies). In some instances, the kits can further contain instructions for methods of using the kit to detect the activation states of a plurality of signal transduction molecules of circulating cells of a solid tumor. The kits may also contain any of the additional reagents described above with respect to performing the specific methods of the present invention such as, for example, first and second members of the signal amplification pair, tyramide signal amplification reagents, GO dextran, wash buffers, etc.

In another embodiment of a two-antibody approach, the present invention provides a method for detecting the presence of a truncated receptor, the method comprising: (a) incubating a cellular extract with a plurality of beads specific for an extracellular domain (ECD) binding region, wherein the ECD binding region is specific for a full-length receptor; (b) removing the plurality of beads from the cellular extract, thereby removing the full length receptors to form a cellular extract devoid of the full length receptors; (c) incubating said cellular extract devoid of said full length receptors with a plurality of capture antibodies, wherein said plurality of capture antibodies are specific for an intracellular domain (ICD) binding region of said truncated receptor and wherein said plurality of captured antibodies are restrained on a solid support to form a plurality of captured truncated receptors; (d) incubating the plurality of captured truncated receptors with detection antibodies specific for the corresponding truncated receptors to form a plurality of detectable captured truncated receptors; (e) incubating the plurality of detectable captured truncated receptors with first and second members of a signal amplification pair to generate an amplified signal; and (f) detecting an amplified signal generated from the first and second members of the signal amplification pair.

In certain embodiments, the truncated receptor is p95 ErbB2 and the full-length receptor is ErbB2 (HER-2). In certain other aspects, the plurality of beads specific for an extracellular domain (ECD) binding region comprise a streptavidin-biotin pair, wherein the biotin is attached to the bead and the biotin is attached to an antibody (e.g., wherein the antibody is specific for the ECD binding region of the full-length receptor).

In another embodiment of a two-antibody approach, the present invention provides a method for detecting the presence of a truncated protein, the method comprising: (a) incubating a cellular extract with a plurality of beads specific for the full-length protein; (b) removing the plurality of beads from the cellular extract, thereby removing the full length protein to form a cellular extract devoid of the full length receptors; (c) incubating said cellular extract devoid of said full length proteins with a plurality of capture antibodies, wherein said plurality of capture antibodies are specific for the truncated protein and wherein said plurality of captured antibodies are restrained on a microcarrier to form a plurality of captured truncated proteins; (d) incubating the plurality of captured truncated proteins with detection antibodies specific for the corresponding truncated proteins to form a plurality of detectable captured truncated proteins; (e) incubating the plurality of detectable captured truncated proteins with first and second members of a signal amplification pair to generate an amplified signal; and (f) detecting an amplified signal generated from the first and second members of the signal amplification pair.

2. Proximity Dual Detection Assays

In some embodiments, the assay for detecting the activation state of a particular analyte (e.g., signal transduction molecule, e.g., AMPK modulator or pathological molecule, e.g., tau, Aβ peptides,and the like) of interest in a cellular extract of cells such as circulating cells is a multiplex, high-throughput proximity (i.e., three-antibody) assay having superior dynamic range. As a non-limiting example, the three antibodies used in the proximity assay can comprise: (1) a capture antibody specific for the analyte; (2) a detection antibody specific for an activated form of the analyte (i.e., activation state-dependent antibody); and (3) a detection antibody which detects the total amount of the analyte (i.e., activation state-independent antibody). The activation state-dependent antibody is capable of detecting, for example, the phosphorylation, ubiquitination, and/or complexation state of the analyte. The activation state-dependent antibody is generally capable of detecting both the activated and non-activated forms of the analyte.

In one embodiment, the proximity assay comprises:
(i) incubating the cellular extract from e.g., cerebral spinal fluid with a plurality of dilution series of capture antibodies against the tau protein to form a plurality of captured tau proteins;
(ii) incubating the plurality of captured tau proteins with detection antibodies comprising a plurality of antibodies against total tau protein (e.g., activation state-independent antibodies) and a plurality of antibodies against phosphorylated tau protein (e.g., activation state-dependent antibodies) to form a plurality of detectable captured activated tau analytes, wherein the antibodies against total tau protein are labeled with a facilitating moiety (e.g., glucose oxidase), the antibodies against phosphorylated tau protein are labeled with a first member of a signal amplification pair (e.g., horseradish peroxidase), and the facilitating moiety (e.g., glucose oxidase) generates an oxidizing agent which channels to and reacts with the first member of the signal amplification pair;
(iii) incubating the plurality of detectable captured activated tau analytes with a second member of the signal amplification pair (e.g., biotin-tyramide) to generate an amplified signal; and
(iv) detecting the amplified signal generated from the horseradish peroxidase and biotin-tyramide using streptavidin-Alexa Fluor® dye as the signal detecting reagent.

In a preferred embodiment, the proximity assay comprises: (i) incubating the cellular extract with a plurality of dilution series of capture antibodies to form a plurality of captured analytes; (ii) incubating the plurality of captured analytes with detection antibodies comprising a plurality of activation state-independent antibodies and a plurality of activation state-dependent antibodies specific for the corresponding analytes to form a plurality of detectable captured analytes, wherein the activation state-independent antibodies are labeled with a facilitating moiety, the activation state-dependent antibodies are labeled with a first member of a signal amplification pair, and the facilitating moiety generates an oxidizing agent which channels to and reacts with the first member of the signal amplification pair; (iii) incubating the plurality of detectable captured analytes with a second member of the signal amplification pair to generate an amplified signal; and (iv) detecting the amplified signal generated from the first and second members of the signal amplification pair.

Alternatively, the activation state-dependent antibodies can be labeled with a facilitating moiety and the activation state-independent antibodies can be labeled with a first member of a signal amplification pair.

The capture antibodies, activation state-independent antibodies, and activation state-dependent antibodies are preferably selected to minimize competition between them with respect to analyte binding (i.e., all antibodies can simultaneously bind their corresponding signal transduction molecules).

In some embodiments, the activation state-independent antibodies further comprise a detectable moiety, In such instances, the amount of the detectable moiety is correlative to the amount of one or more of the analytes in the cellular extract. Examples of detectable moieties include, but are not limited to, fluorescent labels, chemically reactive labels, enzyme labels, radioactive labels, and the like. Preferably, the detectable moiety is a fluorophore such as an Alexa Fluor® dye (e.g., Alexa Fluor® 647), fluorescein, fluorescein isothiocyanate (FITC), Oregon Green™; rhodamine, Texas red, tetrarhodamine isothiocynate (TRITC), a CyDye™ fluor (e.g., Cy2, Cy3, Cy5), and the like. The detectable moiety can be coupled directly or indirectly to the activation state-independent antibodies using methods well-known in the art.

In certain instances, the activation state-independent antibodies are directly labeled with the facilitating moiety. The facilitating moiety can be coupled to the activation state-independent antibodies using methods well-known in the art. A suitable facilitating moiety for use in the present invention includes any molecule capable of generating an oxidizing agent which channels to (i.e., is directed to) and reacts with (i.e., binds, is bound by, or forms a complex with) another molecule in proximity (i.e., spatially near or close) to the facilitating moiety. Examples of facilitating moieties include, without limitation, enzymes such as glucose oxidase or any other enzyme that catalyzes an oxidation/reduction reaction involving molecular oxygen (O₂) as the electron acceptor, and photosensitizers such as methylene blue, rose bengal, porphyrins, squarate dyes, phthalocyanines, and the like. Non-limiting examples of oxidizing agents include hydrogen peroxide (H₂O₂), a singlet oxygen, and any other compound that transfers oxygen atoms or gains electrons in an oxidation/reduction reaction. Preferably, in the presence of a suitable substrate (e.g., glucose, light, etc.), the facilitating moiety (e.g., glucose oxidase, photosensitizer, etc.) generates an oxidizing agent (e.g., hydrogen peroxide (H₂O₂), single oxygen, etc.) which channels to and reacts with the first member of the signal amplification pair (e.g., horseradish peroxidase (HRP), hapten protected by a protecting group, an enzyme inactivated by thioether linkage to an enzyme inhibitor, etc.) when the two moieties are in proximity to each other.

In certain other instances, the activation state-independent antibodies are indirectly labeled with the facilitating moiety via hybridization between an oligonucleotide linker conjugated to the activation state-independent antibodies and a complementary oligonucleotide linker conjugated to the facilitating moiety. The oligonucleotide linkers can be coupled to the facilitating moiety or to the activation state-independent antibodies using methods well-known in the art. In some embodiments, the oligonucleotide linker conjugated to the facilitating moiety has 100% complementarity to the oligonucleotide linker conjugated to the activation state-independent antibodies. In other embodiments, the oligonucleotide linker pair comprises at least one, two, three, four, five, six, or more mismatch regions, e.g., upon hybridization under stringent hybridization conditions. One skilled in the art will appreciate that activation state-independent antibodies specific for different analytes can either be conjugated to the same oligonucleotide linker or to different oligonucleotide linkers.

The length of the oligonucleotide linkers that are conjugated to the facilitating moiety or to the activation state-independent antibodies can vary. In general, the linker sequence can be at least about 5, 10, 15, 20, 25, 30, 35, 40, 45, 50, 75, or 100 nucleotides in length. Typically, random nucleic acid sequences are generated for coupling. As a non-limiting example, a library of oligonucleotide linkers can be designed to have three distinct contiguous domains: a spacer domain; signature domain; and conjugation domain. Preferably, the oligonucleotide linkers are designed for efficient coupling without destroying the function of the facilitating moiety or activation state-independent antibodies to which they are conjugated.

The oligonucleotide linker sequences can be designed to prevent or minimize any secondary structure formation under a variety of assay conditions. Melting temperatures are typically carefully monitored for each segment within the linker to allow their participation in the overall assay procedures. Generally, the range of melting temperatures of the segment of the linker sequence is between 1-10° C. Computer algorithms (e.g., OLIGO 6.0) for determining the melting temperature, secondary structure, and hairpin structure under defined ionic concentrations can be used to analyze each of the three different domains within each linker. The overall combined sequences can also be analyzed for their structural characterization and their comparability to other conjugated oligonucleotide linker sequences, e.g., whether they will hybridize under stringent hybridization conditions to a complementary oligonucleotide linker.

The spacer region of the oligonucleotide linker provides adequate separation of the conjugation domain from the oligonucleotide crosslinking site. The conjugation domain functions to link molecules labeled with a complementary oligonucleotide linker sequence to the conjugation domain via nucleic acid hybridization. The nucleic acid-mediated hybridization can be performed either before or after antibody-analyte (i.e., antigen) complex formation, providing a more flexible assay format. Unlike many direct antibody conjugation methods, linking relatively small oligonucleotides to antibodies or other molecules has minimal impact on the specific affinity of antibodies towards their target analyte or on the function of the conjugated molecules.

In some embodiments, the signature sequence domain of the oligonucleotide linker can be used in complex multiplexed protein assays. Multiple antibodies can be conjugated with oligonucleotide linkers with different signature sequences. In multiplex immunoassays, reporter oligonucleotide sequences labeled with appropriate probes can be used to detect cross-reactivity between antibodies and their antigens in the multiplex assay format.

Oligonucleotide linkers can be conjugated to antibodies or other molecules using several different methods. For example, oligonucleotide linkers can be synthesized with a thiol group on either the 5′ or 3′ end. The thiol group can be deprotected using reducing agents (e.g., TCEP-HCl) and the resulting linkers can be purified by using a desalting spin column. The resulting deprotected oligonucleotide linkers can be conjugated to the primary amines of antibodies or other types of proteins using heterobifunctional cross linkers such as SMCC. Alternatively, 5′-phosphate groups on oligonucleotides can be treated with water-soluble carbodiimide EDC to form phosphate esters and subsequently coupled to amine-containing molecules. In certain instances, the diol on the 3′-ribose residue can be oxidized to aldehyde groups and then conjugated to the amine groups of antibodies or other types of proteins using reductive amination. In certain other instances, the oligonucleotide linker can be synthesized with a biotin modification on either the 3′ or 5′ end and conjugated to streptavidin-labeled molecules.

Oligonucleotide linkers can be synthesized using any of a variety of techniques known in the art, such as those described in Usman et al., J. Am. Chem. Soc., 109:7845 (1987); Scaringe et al., Nucl. Acids Res., 18:5433 (1990); Wincott et al., Nucl. Acids Res., 23:2677-2684 (1995); and Wincott et al., Methods Mol. Bio., 74:59 (1997). In general, the synthesis of oligonucleotides makes use of common nucleic acid protecting and coupling groups, such as dimethoxytrityl at the 5′-end and phosphoramidites at the 3′-end. Suitable reagents for oligonucleotide synthesis, methods for nucleic acid deprotection, and methods for nucleic acid purification are known to those of skill in the art.

In certain instances, the activation state-dependent antibodies are directly labeled with the first member of the signal amplification pair. The signal amplification pair member can be coupled to the activation state-dependent antibodies using methods well-known in the art. In certain other instances, the activation state-dependent antibodies are indirectly labeled with the first member of the signal amplification pair via binding between a first member of a binding pair conjugated to the activation state-dependent antibodies and a second member of the binding pair conjugated to the first member of the signal amplification pair. The binding pair members (e.g., biotin/streptavidin) can be coupled to the signal amplification pair member or to the activation state-dependent antibodies using methods well-known in the art. Examples of signal amplification pair members include, but are not limited to, peroxidases such horseradish peroxidase (HRP), catalase, chloroperoxidase, cytochrome c peroxidase, eosinophil peroxidase, glutathione peroxidase, lactoperoxidase, myeloperoxidase, thyroid peroxidase, deiodinase, and the like. Other examples of signal amplification pair members include haptens protected by a protecting group and enzymes inactivated by thioether linkage to an enzyme inhibitor.

In one example of proximity channeling, the facilitating moiety is glucose oxidase (GO) and the first member of the signal amplification pair is horseradish peroxidase (HRP). When the GO is contacted with a substrate such as glucose, it generates an oxidizing agent (i.e., hydrogen peroxide (H₂O₂)). If the HRP is within channeling proximity to the GO, the H₂O₂ generated by the GO is channeled to and complexes with the HRP to form an HRP-H₂O₂ complex, which, in the presence of the second member of the signal amplification pair (e.g., a chemiluminescent substrate such as luminol or isoluminol or a fluorogenic substrate such as tyramide (e.g., biotin-tyramide), homovanillic acid, or 4-hydroxyphenyl acetic acid), generates an amplified signal. Methods of using GO and HRP in a proximity assay are described in, e.g., Langry et al., U.S. Dept. of Energy Report No. UCRL-ID-136797 (1999). When biotin-tyramide is used as the second member of the signal amplification pair, the HRP-H₂O₂ complex oxidizes the tyramide to generate a reactive tyramide radical that covalently binds nearby nucleophilic residues. The activated tyramide is either directly detected or detected upon the addition of a signal-detecting reagent such as, for example, a streptavidin-labeled fluorophore or a combination of a streptavidin-labeled peroxidase and a chromogenic reagent. Examples of fluorophores suitable for use in the present invention include, but are not limited to, an Alexa Fluor® dye (e.g., Alexa Fluor® 555, Alexa Fluor® 647, etc.), fluorescein, fluorescein isothiocyanate (FITC), Oregon Green™; rhodamine, Texas red, tetrarhodamine isothiocynate (TRITC), a CyDye™fluor (e.g., Cy2, Cy3, Cy5), and the like. The streptavidin label can be coupled directly or indirectly to the fluorophore or peroxidase using methods well-known in the art. Non-limiting examples of chromogenic reagents suitable for use in the present invention include 3,3′,5,5′-tetramethylbenzidine (TMB), 3,3′-diaminobenzidine (DAB), 2,2′-azino-bis(3-ethylbenzothiazoline-6-sulfonic acid) (ABTS), 4-chloro-1-napthol (4CN), and/or porphyrinogen.

In another example of proximity channeling, the facilitating moiety is a photosensitizer and the first member of the signal amplification pair is a large molecule labeled with multiple haptens that are protected with protecting groups that prevent binding of the haptens to a specific binding partner (e.g., ligand, antibody, etc.). For example, the signal amplification pair member can be a dextran molecule labeled with protected biotin, coumarin, and/or fluorescein molecules. Suitable protecting groups include, but are not limited to, phenoxy-, analino-, olefin-, thioether-, and selenoether-protecting groups. Additional photosensitizers and protected hapten molecules suitable for use in the proximity assays of the present invention are described in U.S. Pat. No. 5,807,675. When the photosensitizer is excited with light, it generates an oxidizing agent (i.e., singlet oxygen). If the hapten molecules are within channeling proximity to the photosensitizer, the singlet oxygen generated by the photosensitizer is channeled to and reacts with thioethers on the protecting groups of the haptens to yield carbonyl groups (ketones or aldehydes) and sulphinic acid, releasing the protecting groups from the haptens. The unprotected haptens are then available to specifically bind to the second member of the signal amplification pair (e.g., a specific binding partner that can generate a detectable signal). For example, when the hapten is biotin, the specific binding partner can be an enzyme-labeled streptavidin. Exemplary enzymes include alkaline phosphatase, (3-galactosidase, HRP, etc. After washing to remove unbound reagents, the detectable signal can be generated by adding a detectable (e.g., fluorescent, chemiluminescent, chromogenic, etc.) substrate of the enzyme and detected using suitable methods and instrumentation known in the art. Alternatively, the detectable signal can be amplified using tyramide signal amplification and the activated tyramide either directly detected or detected upon the addition of a signal-detecting reagent as described above.

In yet another example of proximity channeling, the facilitating moiety is a photosensitizer and the first member of the signal amplification pair is an enzyme-inhibitor complex. The enzyme and inhibitor (e.g., phosphonic acid-labeled dextran) are linked together by a cleavable linker (e.g., thioether). When the photosensitizer is excited with light, it generates an oxidizing agent (i.e., singlet oxygen). If the enzyme-inhibitor complex is within channeling proximity to the photosensitizer, the singlet oxygen generated by the photosensitizer is channeled to and reacts with the cleavable linker, releasing the inhibitor from the enzyme, thereby activating the enzyme. An enzyme substrate is added to generate a detectable signal, or alternatively, an amplification reagent is added to generate an amplified signal.

In a further example of proximity channeling, the facilitating moiety is HRP, the first member of the signal amplification pair is a protected hapten or an enzyme-inhibitor complex as described above, and the protecting groups comprise p-alkoxy phenol. The addition of phenylenediamine and H₂O₂ generates a reactive phenylene diimine which channels to the protected hapten or the enzyme-inhibitor complex and reacts with p-alkoxy phenol protecting groups to yield exposed haptens or a reactive enzyme. The amplified signal is generated and detected as described above (see, e.g., U.S. Pat. Nos. 5,532,138 and 5,445,944).

In another embodiment, the present invention provides kits for performing the proximity assays described above comprising: (a) a dilution series of a plurality of capture antibodies restrained on a microcarrier; and (b) a plurality of detection antibodies (e.g., activation state-independent antibodies and activation state-dependent antibodies). In some instances, the kits can further contain instructions for methods of using the kit to detect the activation states of a plurality of signal transduction molecules of circulating cells of a solid tumor. The kits may also contain any of the additional reagents described above with respect to performing the specific methods of the present invention such as, for example, first and second members of the signal amplification pair, tyramide signal amplification reagents, substrates for the facilitating moiety, wash buffers, etc.

B. Microcarriers

In the present invention the analyte capture and detection assay described above, in particular the capture antibody, is coupled to a microcarrier. Detailed descriptions of microcarriers useful in the invention are found in, e.g., International Appl. Publ. No. WO 2012/106827 and U.S. Patent Publ. Nos. 2011/0306506 and 2013/0095574, which are herein incorporated by reference in their entirety for all purposes.

In certain aspects, the microcarrier provided is a solid support in the shape of a sphere, wafer, square, disk, rectangle, circle, triangle, hexagon or polygon. In some embodiments, the height of the microcarrier is smaller than both its width and its height. In some embodiments, the microcarrier has two essentially parallel and essentially flat surfaces at the top and the bottom.

In some embodiments, the microcarrier has a disk-like shape (e.g., a wafer shape) with a circle-shaped detection surface. In some instances, the body of the microcarrier has at least a spacing element projecting from the body and shaped to ensure that when the encoded microcarrier is positioned on a flat plane with the detection surface facing the flat plane, a gap exists between the flat plane and the detection surface.

In some embodiments, the diameter of the disk-like shape is between about 1 to about 300 μm such as 1, 10, 20, 30, 40, 50, 60, 70, 80, 90, 100, 125, 150, 175, 200, 225, 250, 275, or 300 μm.

The microcarrier can be identified by a code such as a digital code which can be a configuration of transversing holes in the microcarrier. A set (e.g., population) of microcarriers is composed of individual microcarriers with the same code and having an identical function. Two different sets of microcarriers can have different functions and different codes.

The code can be any pattern of transversing holes that pass through the entire thickness of the microcarrier including the detection surface. In some instances, the code also includes an asymmetric orientation mark such as a triangle or an L-shaped sign.

In some embodiments, the code is determined by reading (imaging) the code. The code can be identified using standard imaging and decoding techniques. The code is preferably used to identify the function of the microcarrier.

In some embodiments, microcarriers of a set have the same function, such as capturing and/or detecting a specific analyte. For instance, a set of microcarriers is composed of a plurality of individually encoded microcarriers associated with a specific target analyte. In certain aspects, the function of the microcarrier corresponds to identifying the capture antibody or capture ligand attached to its surface.

Microcarriers can be made from any material routinely used in high-throughput screening technology and diagnostics. Non-limiting examples of these materials include latex, polystyrene, cross-linked dextrans, polymethylstyrene, polycarbonate, polypropylene, cellulose, polyacrylamide, polydimethylacrylamide, fluorinated ethylene-propylene as well as materials commonly used in microfabrication or micromilling such as glass, SiO₂, silicon, PMMA (polymethylmethacrylate), gold, silver, aluminium, steel or other metals or epoxy-based photosensitive materials such as SU-8. The microcarriers may be of any shapes and sizes. In some embodiments, the microcarriers are made of silicon.

The microcarriers can be biofunctionalized with one or more capture antibodies of the invention. Any known method can be used to attach the capture antibody to the microcarrier.

C. Diagnostic System

In the diagnostic system provided herein encoded microcarriers are placed in the microchannel of the assay device of a microfluidic instrument. In some embodiments, the microchannel serves as a reaction chamber comprising at least one or more sets of microcarriers. Typically, the microchannel is transparent on at least one side to enable detection of fluorescence emitted by the microcarriers. In some embodiments, the microfluidic channel has a transparent observation wall.

The term “microfluidic channel” or “microchannel” refers to a closed channel (e.g., an elongated passage for fluids), with a cross-section being from about 1 to about 500 μm, preferably about 10 to about 300 μm such as 1, 10, 20, 30, 40, 50, 60, 70, 80, 90, 100, 125, 150, 175, 200, 225, 250, 275, or 300 μm. A microfluidic channel has a longitudinal direction, that is not necessarily a straight line, and that corresponds to the direction in which fluids are directed within the microfluidic channel. Preferably, the direction corresponds to the average speed vector of the fluid, thereby assuming a laminar flow regime.

The microchannel is designed to allow the microcarriers to form a monolayer over the entire length of the microchannel, to limit the volume around the microcarriers to ensure that the sample passes by a maximum number of microcarriers, and to restrict the movement of the microcarriers along the length of the microchannel while allowing the sample and reagent fluids to flow through.

The microchannel can be made of material including, but not limited to, silicon, SU-8 (an epoxy based photoresist), polyimide (PI), polydimethylsiloxane (PDMS), silicone, or other thermoplastic elastomers (TPE), polymethylmethacrylate (PMMA), Teflon (PTFE), thermoplastic elastomers (TPE), Victrex PEEK™, Polycarbonate, Polyvinyl chloride (PVC), polypropylene (PP), polyethylene (PE), polystyrene (PS), Fluorinated Ethylene-Propylene (FEP), Cyclic Olefin (Co)polymer (COP or COC) or other thermoplastic polymers, quartz, glass or plateable metals such as nickel, silver or gold, most preferably of transparent polymers.

The microcarriers of the assay device have a shape relative to the cross-section of the microchannel that allows having, over the entire length of the microchannel, at least two of any of the microcarriers arranged side-by-side without touching each other and without touching the perimeter of the microchannel.

D. Analytes

Non-limiting examples of analytes such as signal transduction molecules that can be interrogated for expression (e.g., total amount) levels and/or activation (e.g., phosphorylation) levels in a cellular extract include receptor tyrosine kinases, non-receptor tyrosine kinases, tyrosine kinase signaling cascade components, nuclear hormone receptors, nuclear receptor coactivators, nuclear receptor repressors, ligands, receptors, signal cascade components, and a combination thereof. Additional examples of analytes include, but are not limited to, include autoantibodies, neurodegenerative molecules, and pathological molecules.

In one embodiment, the methods of the present invention comprise determining the presence, expression level (e.g., total amount) and/or activation level (e.g., level of phosphorylation or complex formation) of at least one or more of the following analytes in a cellular extract: HER1, HER2, HER3. HER4, VEGFR-1, VEGFR-2, VEGFR-3, FLT-3, FLK-2, PDGR, c-KIT, INSR, IGF-IR, IGF-IIR, IRR, CSF-IR, cMET, and a combination thereof.

In another embodiment, the methods of the present invention comprise determining the presence, expression level (e.g., total amount) and/or activation level (e.g., level of phosphorylation or complex formation) of at least one or more of the following analytes in a cellular extract: AMP-activated protein kinase (AMPK), AMPK kinase (AMPKK), calmodulin-dependent protein kinase kinase (CAMKK), LKB1, transforming growth factor-β-activated kinase 1 (TAK1), AKT, adiponectin, leptin, glucose, other AMP modulators, and a combination thereof.

In yet another embodiment, the methods of the present invention comprise determining the presence and/or the expression level (e.g., total amount) of at least one or more autoantibodies such as a GAD65 autoantibody, insulinoma-antigen 2 (IA-2) autoantibody, insulin autoantibody (IAA), zinc transporter protein 8 (ZnT8) autoantibody and a combination thereof.

Glutamic acid decarboxylase 65 (GAD65) is a neuronal enzyme involved in the synthesis of gamma-aminobutyric acid (GABA). It has been shown that the GAD65 autoantibody can be used as a biomarker of autoimmune disease associated with type I diabetes (Atkinson et al., Lancet, 335:1357-60 (1990)). Furthermore, GAD65, IA-2, IAA, ZnT8 autoantibodies have been proposed to be predictive of type 1 diabetes (Mueller et al., Clinical Laboratory News, 36(10), October 2010; Bingley, P J., J. Clin. Endocrinol. Metab., 95(1):25-33 (2010)).

In another embodiment, the methods of the present invention comprise determining the presence, the expression level (e.g., total amount) and/or activation level (e.g., level of phosphorylation or complex formation) of at least one or more of the following analytes in a cellular extract: tau, amyloid beta (Aβ) peptides (e.g., β-amyloid protein 42), soluble amyloid precursor protein beta (sAPPP), VILIP-1 and a combination thereof. The concentrations of several analytes, such as β-amyloid protein 42 (Aβ42), sAPPβ, total tau protein, and phosphorylated tau, have been associated with Alzheimer's disease.

In one particular embodiment, the present invention comprises determining the presence, expression level (e.g., total amount) and/or activation level (e.g., level of phosphorylation or complex formation) of at least one, two, three, four, five, six, seven, eight, nine, ten, or more analytes in a multiplex assay.

In certain embodiments, the present invention further comprises determining the expression level (e.g., total amount) and/or activation level (e.g., level of phosphorylation or complex formation) of one or more (e.g., at least about 1, 2, 3, 4, 5, 6, 7, 8, 9, 10, 11, 12, 13, 14,15, 16, 17, 18, 19, 20, 21, 22, 23, 24, 25, 26, 27, 28, 29, 30, 35, 40, 45, 50, or more) additional analytes in a cellular extract. In some embodiments, the one or more (e.g., at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more) additional analytes comprises one or more signal transduction molecules selected from the group consisting of receptor tyrosine kinases, non-receptor tyrosine kinases, tyrosine kinase signaling cascade components, nuclear hormone receptors, nuclear receptor coactivators, nuclear receptor repressors, ligands, receptors, signal cascade components; one or more neurodegenerative proteins; one or more pathological proteins; and a combination thereof.

### IV. EXAMPLES

The following examples are offered to illustrate, but not to limit, the claimed invention.

### Example 1

### Microfluidic CEER for Measuring Phosphorylated HER2 Levels in a Patient Sample

This example illustrates a method of detecting activated HER2 levels in cellular lysate samples using proximity dual detection assays (e.g., CEER assays) coupled microcarriers and a microfluidics-based instrument.

The objective of this study was to determine the optimal conditions for measuring an activated analyte in a sample derived from a cell. The parameters that were tested included the amount (e.g., concentration) of the capture antibody, the amount of sample, and the amount (e.g., dilution) of the second member of the signal amplification pair.

HER2 capture antibodies at a concentration of 0.5 mg/ml and 1.0 mg/ml were coupled onto barcoded microcarriers. IgG which served as a negative control for the assay was also coupled to another set of microcarriers. Next, the coupled, encoded microcarriers were blocked with 5% BSA in PBST for one hour and then loaded onto the assay cartridge (assay device).

Next, cell lysate samples from HER2 overexpressing cells (i.e., BT474 cells) were loaded onto the cartridge and incubated with the coupled microcarriers in the reaction chamber for 80 minutes. The samples tested included lysates corresponding to 75 cells (e.g., 100 cells in 80 μl volume), 7.5 cells (e.g., 10 cells in 80 μl volume) and 0.75 cells (e.g., 1 cells in 80 μl volume). After the incubation period, the samples were removed through the outlet port by the microfluidic instrument.

The detection antibodies (e.g., 4G10-HRP and HER2-AB4-GO) were incubated with the coupled microcarriers for 20 minutes and were removed by the microfluidic instrument. The antibody 4G10-HRP specifically binds phosphotryosine and the antibody HER2-AB4-GO recognizes the HER2 polypeptide.

Next, biotin-tyramide (BT-tyr), the second member of the signal amplification pair, was added to the reaction chamber, incubated for 30 minutes and removed from the reaction chamber. Two dilutions (1:500 and 1:1000) of biotin-tyramide were tested. It was determined that higher concentrations (e.g., levels) of biotin-tyramide generated fluorescent aggregates which interfered with the fluorescent readout. The signal detection reagent streptavidin-Alexa647 at a concentration of 0.5 μg/ml was added and incubated for 5 minutes.

The fluorescence of the microcarriers was detected and quantitated using standard microscopic imaging technology and fluorescence analysis software.

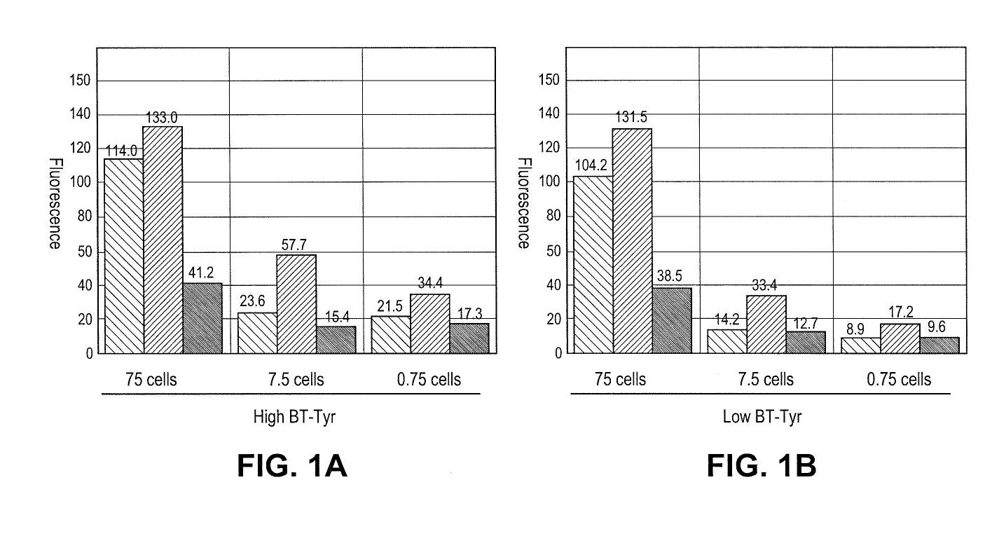

The results show that activated HER2 levels were detected and quantitated in samples containing as few as 0.75 cells (FIGS. 1A and 1B). In FIG. 1A, the dilution series with high BT-Tyr shows the fluorescence intensity left to right of Anti-HER2 (0.5 mg/mL; 114), Anti-HER2 (1.0 mg/mL;133) and IgG (negative control) (1.0 mg/mL; 41.2). In FIG. 1B, the dilution series with low BT-Tyr shows the fluorescence intensity left to right of Anti-HER2 (0.5 mg/mL; 104.2), Anti-HER2 (1.0 mg/mL;131.5) and IgG (negative control) (1.0 mg/mL; 38.5). Moreover, HER2 levels were detectable at both concentrations of either the capture antibody and biotin-tyramide. The data also shows that the sensitivity of the microcarrier-based assay was comparable to traditional CEER assays that employ capture antibodies coupled to nitrocellulose-coated glass slides.

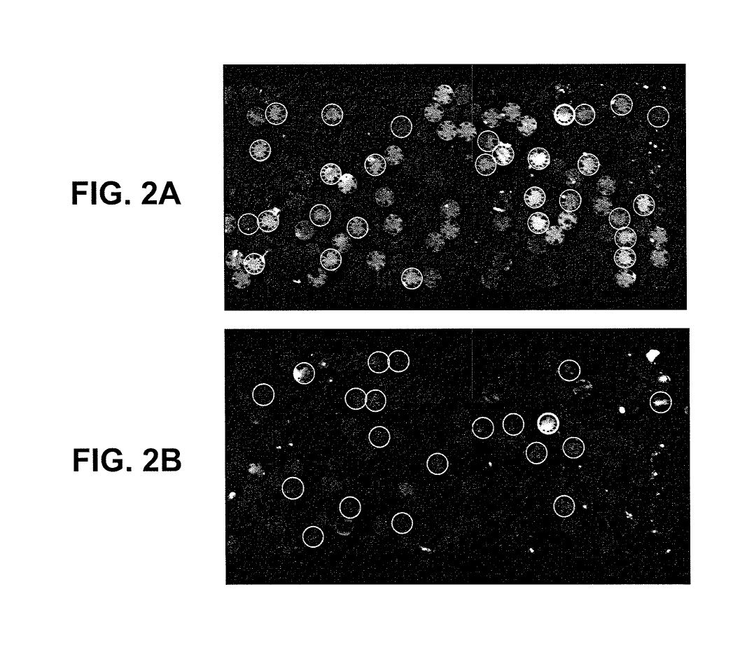

FIG. 2 shows that the signal to background ratio decreased when fewer cells were loaded onto the reaction chamber. In particular, FIG. 2A shows fluorescent microcarriers in the assay device that were incubated with a sample of 75 BT474 cells. FIG. 2B shows similar fluorescent microcarriers incubated with a sample of 7.5 cells.

This example illustrates a method of detecting activated HER2 levels in cellular lysate samples using proximity dual detection assays (e.g., CEER assays) coupled microcarriers and a microfluidics-based instrument.

### Example 2

### Microfluidic CEER for Measuring Phosphorylated Tau Levels in a Patient Sample

This example describes a method for using the microfluid CEER assay described herein to measure the level of phosphorylated tau in a sample derived from a patient. The level of activated tau can be used to determine whether the patient has Alzheimer's disease (AD) or has an increased risk of developing AD.

A sample of cerebrospinal fluid is taken from the patient via lumbar puncture according to standard clinical protocols. The cells of the sample are lysed using a standard lysate buffer to generate a cellular extract.

The capture antibody that recognizes non-phosphorylated tau is attached to a set of encoded microcarriers using a standard coupling procedure such as Biocartis' capture molecule coupling protocol. The coupled microcarriers are blocked with a 5% BSA solution in PBST for one hour. The coupled microcarriers are loaded into the reaction chamber of the assay cartridge using a microfluidic instrument.

The cellular extract is loaded into the reaction chamber and allowed to contact the coupled microcarriers for about 80 minutes. The cellular extract is removed by laminar fluid flow and the detection antibodies are loaded into the reaction chamber. The detection antibodies include a first antibody against non-phosphorylated tau and coupled to glucose oxidase and a second antibody against phosphorylated tau and coupled to horseradish peroxidase. The detection antibodies are incubated with the captured tau proteins attached to the microcarriers for about 20 minutes and are then removed. Next, biotin-tyramide is loaded into the reaction chamber containing the detected captured tau proteins attached to the microcarrier. The signal amplification reaction is carried out for about 30 minutes. The signal detection reagent streptavidin-AlexaFluor® 647 is then incubated with the detected captured analytes for 5 minutes. Finally, the fluorescence signal from the microcarriers is detected using a CCD camera and quantitated using standard imaging software. In addition, the code on the microcarriers is read to decipher the function of the microcarriers.

The measured fluorescence corresponds to the level of the target analyte (e.g., activated tau) present in the sample. If the level of phosphorylated tau is elevated in a patient sample compared to a non-AD control, then it is determined that the patient has AD or is at risk of developing AD.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

1. A method for performing a multiplexed immunoassay on a sample, the method comprising:
(a) contacting a cell lysate with a plurality of capture antibodies attached to an encoded microcarrier, specific for at least one analyte to form a plurality of captured analytes;
(b) contacting the plurality of captured analytes with at least two types of detection antibodies specific for the corresponding analytes, wherein at least one type of detection antibodies has a first member of a signal amplification pair, to form a plurality of detectable captured analytes;
(c) contacting the plurality of detectable captured analytes with a second member of the signal amplification pair to generate an amplified signal; and
(d) detecting the amplified signal generated from the first and second members of the signal amplification pair.

2. The method of claim 1, wherein the encoded microcarrier is made from a material selected from the group consisting of latex, polystyrene, cross-linked dextrans, polymethylstyrene, polycarbonate, polypropylene, cellulose, polyacrylamide, polydimethylacrylamide, fluorinated ethylene-propylene, glass, SiO₂, silicon, PMMA (polymethylmethacrylate), gold, silver, aluminum, steel, and SU-8.

3. The method of claim 1, wherein the encoded microcarrier is made of silicon.

4. The method of claim 1, wherein the encoded microcarrier has a function, which is determined by reading the code.

5. The method of claim 1, wherein the encoded microcarrier is shaped as a member selected from the group consisting of a sphere, a wafer, a square, a disk, a rectangle, a circle, a triangle and a hexagon.

6. The method of claim 1, wherein the encoded microcarrier is shaped as a wafer.

7. The method of claim 1, wherein the encoded microcarrier comprises a plurality of microcarriers.

8. The method of claim 7, wherein the plurality of microcarriers has at least two populations.

9. The method of claim 8, wherein each of the at least two populations of encoded microcarriers has different functions.

10. The method of claim 9, wherein the specific function is identifiable by the code.

11. The method of claim 1, wherein the code is in the form of a configuration of traversing holes in the microcarrier.

12. The method of claim 9, wherein the specific function is the identity of the plurality of capture antibodies.

13. The method of claim 1, wherein the sample is selected from the group consisting of whole blood, serum, plasma, urine, sputum, bronchial lavage fluid, tears, nipple aspirate, lymph, saliva, fine needle aspirate (FNA), cerebral spinal fluid, and a combination thereof.

14. The method of claim 1, wherein the at least one analyte comprises at least one signal transduction molecule, a pathological molecule, or an autoantibody.

15. The method of claim 1, wherein the detection antibodies comprise:
(i) a plurality of activation state-independent antibodies labeled with a facilitating moiety, and
(ii) a plurality of activation state-dependent antibodies labeled with the first member of a signal amplification pair.

16. The method of claim 15, wherein the facilitating moiety generates an oxidizing agent which channels to and reacts with the first member of the signal amplification pair.

17. The method of claim 15, wherein the facilitating moiety is glucose oxidase.

18. The method of claim 16, wherein the oxidizing agent is hydrogen peroxide (H₂O₂).

19. The method of claim 1, wherein the first member of the signal amplification pair is a peroxidase.

20-45. (canceled)

46. A point of care diagnostic system, the system comprising:
(a) an assay device comprising a reaction chamber, wherein the reaction chamber comprises a microchannel; and
b) a kit comprising:
i) a first population of individually encoded microcarriers having a function, wherein the first population of individually encoded microcarriers has a first plurality of capture antibodies attached thereto; and
ii) at least two types of detection antibodies comprising;
a) a plurality of activation state-independent antibodies labeled with a facilitating moiety; and
b) a plurality of activation state-dependent antibodies labeled with a first member of a signal amplification pair.

47.-58. (canceled)
